(19) United States
(12) Patent Application Publication
(10) Pub. No.: US 2024/0418440 A1
(43) Pub. Date: Dec. 19, 2024

(54) CONTAINER DRYING AND HEATING BASE

(71) Applicant: Zhejiang lianhong Technology Co., LTD, Huzhou (CN)

(72) Inventor: Xiu CHEN, Hangzhou (CN)

(73) Assignee: Zhejiang lianhong Technology Co., LTD, Huzhou (CN)

(21) Appl. No.: 18/787,645

(22) Filed: Jul. 29, 2024

(30) Foreign Application Priority Data
Jun. 13, 2023 (CN) 2023215058974

(51) Int. Cl. F26B 3/04; A61L 2/20; F26B 25/00

(52) U.S. Cl. CPC F26B 3/04; A61L 2/202; F26B 25/003; A61L 2202/23

(57) ABSTRACT

Provided in the utility model is a container drying and heating base, including: a supporting column with a hollow structure inside; a heating component located below the supporting column; and a blowing component located below the heating component, where the supporting column is configured to insert an external container to be dried, the heating component is configured to heat air, and the blowing component is configured to blow the air into an interior of the external container; and the supporting column has a thin upper end and a thick lower end, and is provided with a circular opening at a top and a plurality of strip-shaped openings on a periphery.

### CROSS-REFERENCE TO RELATED APPLICATIONS

The application claims priority to Chinese patent application No. 2023215058974, filed on Jun. 13, 2023, the entire contents of which are incorporated herein by reference.

### TECHNICAL FIELD

The utility model relates to the technical field of cleaning equipment, specifically to a container drying and heating base.

### BACKGROUND

Containers have poor air circulation inside due to their storage space, making it difficult to dry the containers after cleaning, especially for soft containers, which can generally be washed with water and used repeatedly; because of the above-mentioned structure, the problem that the inner wall is not easy to be dried after washing occurs, so that the inner wall and the space wrapped therein can easily become a damp closed environment; and breeding bacteria and mildew spots causes harm to people's health and, at the same time, damages the service life of the containers.

Therefore, there is a need to provide a device to solve the above-mentioned problems.

### SUMMARY

In response to the problems existing in the prior art, the utility model provides a drying and heating base.

To achieve the above purpose, the specific solution of the utility model is as follows:
the utility model provides a container drying and heating base, including:
a supporting column with a hollow structure inside;
a heating component located below the supporting column; and
a blowing component located below the heating component,
where the supporting column is configured to insert an external container to be dried, the heating component is configured to heat air, and the blowing component is configured to blow the air into an interior of the external container to dry the external container; and
the supporting column has a thin upper end and a thick lower end, and is provided with a circular opening at a top and a plurality of strip-shaped openings on a periphery.

Furthermore, the supporting column is disposed on an upper shell; and
the upper shell is bowl-shaped.

Furthermore, the upper shell is snap-fitted on a lower shell, and the lower shell is also bowl-shaped.

Furthermore, the upper shell and the lower shell are provided with a plurality of water-filtering holes that are vertically through.

Furthermore, the heating component includes: a holder, a heating rod and a heating wire; and
the holder is installed on the lower shell, a lower end of the heating rod is installed on the holder, an upper end of the heating rod is inserted into the supporting column, and the heating wire is wound around the heating rod.

Furthermore, the blowing component includes a fan; and
the fan is located below the holder and is configured to blow hot air heated by the heating component into an inner side of the external container.

Furthermore, a right side of the holder is further provided with a control mainboard between the upper shell and the lower shell; and
a button is further disposed at a position of a side wall of the lower shell corresponding to the control mainboard.

Furthermore, an inner wall of the upper shell is further provided with a positioning groove, and the positioning groove is wide at a top and narrow at a bottom.

Furthermore, the side wall of the lower shell is further provided with a power supply interface.

Furthermore, the control mainboard is electrically connected with the button, the power supply interface, the heating wire, and the fan each; and an ozone generator is further provided between the upper shell and the lower shell, and ozone is sent to an inner surface of the external container by the fan within a set time for disinfection of the inner surface.

Adopting the technical solution of the utility model has the following beneficial effects:

1. the supporting column has a hollow structure inside, with a thin upper end and a thick lower end for easy placement of the external container;

2. the supporting column is provided with a circular opening at a top and a plurality of strip-shaped openings on a periphery to facilitate water filtration;

3. a heating rod and a heating wire are installed inside the supporting column, so as to penetrate deep into an inner side of the external container for heating; and

4. the fan is located below the heating component, drying can be performed by blowing air alone, and hot air can also be blown in when heated by the heating assembly to accelerate drying and heating.

### BRIEF DESCRIPTION OF DRAWINGS

FIG. 1 is a stereoscopic view of the utility model;

FIG. 2 is an exploded view of the utility model; and

FIG. 3 is a stereoscopic view of an upper shell of the utility model.

In the figures:

1. supporting column; 2. upper shell; 3. lower shell; 4. holder; 5. heating rod; 6. heating wire; 7. fan; 8. control mainboard; 9. button; 10. positioning groove; 11. power supply interface; and 12. water-filtering hole.

### DETAILED DESCRIPTION OF THE EMBODIMENTS

A further detailed explanation of the utility model will be provided below in conjunction with the accompanying drawings and embodiments. It can be understood that the specific embodiments described here are only used to explain the utility model, and not to limit the utility model. Additionally, it should be noted that for ease of description, only a portion related to the utility model is shown in the accompanying drawings, rather than the entire structure.

In the description of the utility model, unless otherwise specified and limited, the term “linkage,” “connection” or “fixation” should be understood in a broad sense, and for example, it can be a fixed connection, a detachable connection, or integrated; it can be a mechanical connection or an electrical connection; and it can be a direct linkage or an indirect linkage through an intermediate medium, and it can be an internal communication of two elements or an interaction relationship between two elements. For one skilled in the art, the specific meanings of the above terms in the utility model can be understood in specific situations.

In the utility model, unless otherwise specified and limited, a first feature “above” or “below” a second feature may include the first and second features in direct contact, or may include the first and second features in contact through another feature between the both instead of direct contact. Moreover, a first feature “on,” “above” and “over” a second feature includes the first feature directly above and diagonally above the second feature, or simply indicates that the first feature is horizontally higher than the second feature. A first feature “under,” “below” and “underneath” a second feature includes the first feature directly below and diagonally below the second feature, or simply indicates that the first feature is horizontally lower than the second feature.

In the description of the embodiment, the terms “up,” “down,” “front,” “back,” “left,” “right” and other directional or positional relationships are based on the directional or positional relationships shown in the accompanying drawings, and only for the convenience of description and simplification of operation, and do not indicate or imply that the device or component referred to must have a specific orientation, be constructed and operated in a specific orientation, and therefore cannot be understood as a limitation to the utility model. In addition, the terms “first” and “second” are only used to distinguish in description and do not have any special meaning.

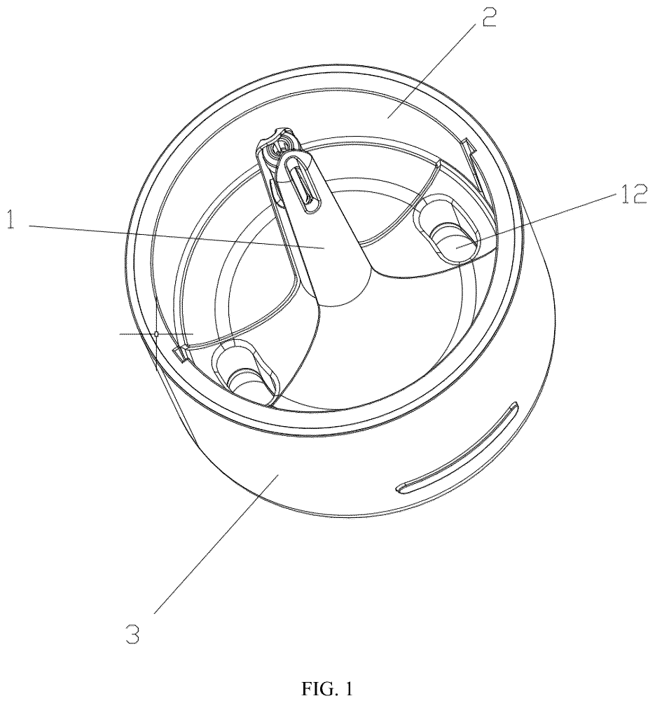

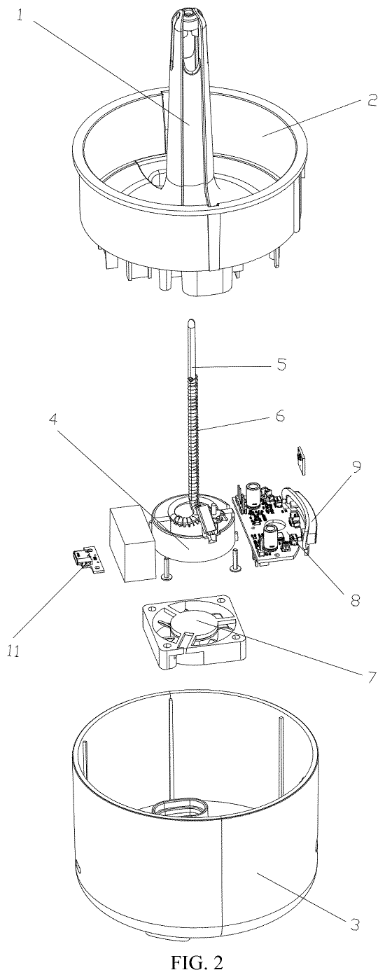

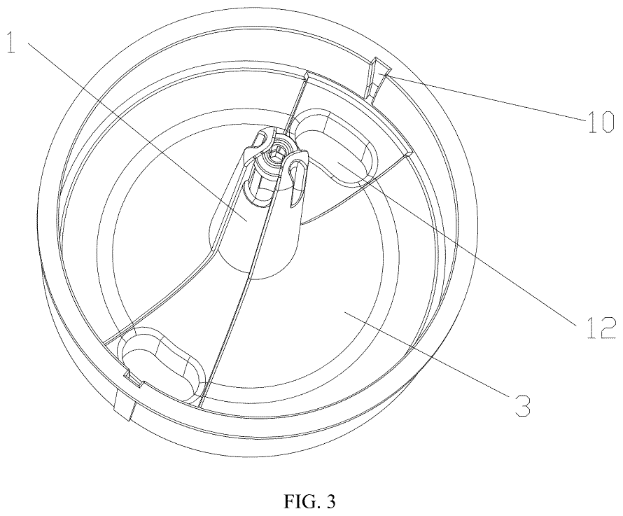

As shown in FIGS. 1-3, the utility model provides a container drying and heating base, including: a supporting column 1 with a hollow structure inside; a heating component located below the supporting column 1; a blowing component located below the heating component, wherein the supporting column 1 is configured to insert an external container to be dried, the heating component is configured to heat the external container, and the blowing component is configured to blow air into an interior of the external container to dry the external container; and the supporting column has a thin upper end and a thick lower end, and is provided with a circular opening at a top and a plurality of strip-shaped openings on a periphery.

The supporting column 1 is disposed on an upper shell 2; and the upper shell 2 is bowl-shaped. The upper shell 2 is snap-fitted on a lower shell 3, and the lower shell 3 is also bowl-shaped. The upper shell 2 and the lower shell 3 are provided with a plurality of water-filtering holes 12 that are vertically through.

The heating component includes: a holder 4, a heating rod 5 and a heating wire 6; and the holder 4 is installed on the lower shell 3, a lower end of the heating rod 5 is installed on the holder 4, an upper end of the heating rod 5 is inserted into the supporting column 1, and the heating wire 6 is wound around the heating rod 5.

The blowing component includes a fan 7; and the fan 7 is located below the holder 4 and is configured to blow hot air heated by the heating component into an inner side of the external container.

A right side of the holder 4 is further provided with a control mainboard 8 between the upper shell 2 and the lower shell 3; and a button 9 is further disposed at a position of a side wall of the lower shell 3 corresponding to the control mainboard 8. An inner wall of the upper shell 2 is further provided with a positioning groove 10, and the positioning groove 10 is wide at a top and narrow at a bottom. The side wall of the lower shell 3 is further provided with a power supply interface 11. The control mainboard 8 is electrically connected to the button 9, the power supply interface 11, the heating wire 6, and the fan 7 each; and an ozone generator is further provided between the upper shell 2 and the lower shell 3, and ozone is sent to an inner surface of the external container by the fan 7 within a set time for disinfection of the inner surface.

The principle of the utility model is as follows:
the supporting column 1 is inserted into the external container to be dried, and water inside the external container flows down and flows out through the water-filtering holes 12 provided on the upper shell 2 and the lower shell 3; the supporting column 1 has a thin upper end and a thick lower end, and is provided with a circular opening at a top and a plurality of strip-shaped openings on a periphery; the holder 4 is installed on the lower shell 3, a lower end of the heating rod 5 is installed on the holder 4, an upper end of the heating rod 5 is inserted into the supporting column 1, and the heating wire 6 is wound around the heating rod 5; and the fan 7 is located below the holder 4 and is configured to blow hot air heated by the heating component into an inner side of the external container.

The above description is only a preferred embodiment of the utility model and does not therefore limit the scope of the patent scope of the utility model. Any equivalent structural transformation made under the utility model concept of the utility model using the specification and drawing content of the utility model, or directly/indirectly applied in other related technical fields are included in the scope of protection of the utility model.

What is claimed is:

1. A container drying and heating base, comprising:
a supporting column with a hollow structure inside;
a heating component located below the supporting column; and
a blowing component located below the heating component,
wherein the supporting column is configured to insert an external container to be dried, the heating component is configured to heat air, and the blowing component is configured to blow the air into an interior of the external container to dry the external container; and
the supporting column has a thin upper end and a thick lower end, and is provided with a circular opening at a top and a plurality of strip-shaped openings on a periphery.

2. The container drying and heating base according to claim 1, wherein the supporting column is disposed on an upper shell; and the upper shell is bowl-shaped.

3. The container drying and heating base according to claim 2, wherein the upper shell is snap-fitted on a lower shell, and the lower shell is also bowl-shaped.

4. The container drying and heating base according to claim 3, wherein the upper shell and the lower shell are provided with a plurality of water-filtering holes that are vertically through.

5. The container drying and heating base according to claim 4, wherein the heating component comprises: a holder, a heating rod and a heating wire; and
the holder is installed on the lower shell, a lower end of the heating rod is installed on the holder, an upper end of the heating rod is inserted into the supporting column, and the heating wire is wound around the heating rod.

6. The container drying and heating base according to claim 5, wherein the blowing component comprises a fan; and
the fan is located below the holder and is configured to blow hot air heated by the heating component into an inner side of the external container.

7. The container drying and heating base according to claim 5, wherein a right side of the holder is further provided with a control mainboard between the upper shell and the lower shell; and
a button is further disposed at a position of a side wall of the lower shell corresponding to the control mainboard.

8. The container drying and heating base according to claim 2, wherein an inner wall of the upper shell is further provided with a positioning groove, and the positioning groove is wide at a top and narrow at a bottom.

9. The container drying and heating base according to claim 7, wherein the side wall of the lower shell is further provided with a power supply interface.

10. The container drying and heating base according to claim 9, wherein the control mainboard is electrically connected with the button, the power supply interface, the heating wire, and the fan each; and
an ozone generator is further provided between the upper shell and the lower shell, and ozone is sent to an inner surface of the external container by the fan within a set time for disinfection of the inner surface.
